(19) United States
(12) Patent Application Publication
(10) Pub. No.: US 2025/0180505 A1
(43) Pub. Date: Jun. 5, 2025

(54) GLUCOSE BIOSENSOR ENCASEMENT, GLUCOSE BIOSENSOR PACKAGE, AND METHOD

(71) Applicant: Medtronic MiniMed, Inc., Northridge, CA (US)

(72) Inventors: Robert Mucic, Glendale, CA (US); Zhenzhong Sun, Northridge, CA (US); Akhil Srinivasan, Woodland Hills, CA (US)

(73) Assignee: Medtronic MiniMed, Inc., Northridge, CA (US)

(21) Appl. No.: 19/052,820

(22) Filed: Feb. 13, 2025

Related U.S. Application Data
(62) Division of application No. 16/691,403, filed on Nov. 21, 2019, now Pat. No. 12,247,941.

(51) Int. Cl. G01N 27/327; A61B 5/145; G01N 27/31

(52) U.S. Cl. CPC G01N 27/3273; A61B 5/14532; G01N 27/31; A61B 2560/0406; A61B 2562/16; A61B 2562/18

(57) ABSTRACT

A glucose biosensor encasement includes a first membrane and a second membrane. The first membrane has a first interlocking segment. The second membrane has a second interlocking segment cooperating with the first interlocking segment of the first membrane to provide a cavity between the first membrane and the second membrane configured to receive a glucose sensor. At least one of the first membrane and the second membrane comprises a semi-permeable portion configured to regulate diffusion characteristics of glucose through the membrane to realize a sensitivity for a sensor in the cavity. A method is also provided.

### CROSS REFERENCE TO RELATED APPLICATION

This application is a divisional application which claims the benefit under 35 U.S.C. § 121 of U.S. patent application Ser. No. 16/691,403, filed Nov. 21, 2019, the contents of which are incorporated herein by reference.

### TECHNICAL FIELD

The subject matter of this application pertains to detection and concentration of analytes in mammals. More particularly, the subject matter relates to the detection and concentration measurement of glucose within a mammal.

### BACKGROUND OF THE DISCLOSURE

Mammals are known to have cellular sensors that self-detect chemical constituents of the body, in particular tissues or blood, so that the body can control changes in constituent concentrations. However, disease and pathophysiological states can impart deviations from normal concentrations of constituents in bodily tissues and blood. Techniques are known for detecting blood glucose levels associated with diabetes. Further similar bodily malfunctions are also desired to be detected so that therapy and diagnostics can be implemented for patients. However, further improvements are needed to increase accuracy and longevity of inserted sensors when measuring constituents and physical membrane lamination and permeability issues can negatively affect performance.

### SUMMARY

A sensor encasement and package is provided for measuring analytes, such as glucose, in the body of mammals that improves reliability and useable life over previously known techniques. Interlocking segments are provided near sensing elements and/or along membrane edges to prevent peeling up of edge layers that could otherwise negatively influence side diffusion of components, or analytes, like glucose that would reduce sensor accuracy and calibration.

In one aspect, glucose biosensor encasement includes a first membrane and a second membrane. The first membrane has a first interlocking segment. The second membrane has a second interlocking segment cooperating with the first interlocking segment of the first membrane to provide a cavity between the first membrane and the second membrane configured to receive a glucose sensor. At least one of the first membrane and the second membrane comprises a semi-permeable portion configured to regulate diffusion characteristics of glucose through the membrane to realize a sensitivity for a sensor in the cavity.

In another aspect, a glucose biosensor package is provided having a first membrane and a second membrane. The first membrane has a first interlocking portion. The second membrane has a second interlocking portion cooperating with the first interlocking portion of the first membrane to provide a cavity between the first membrane and the second membrane configured to receive a biosensor. The biosensor has at least one sensing element contained in the cavity. At least one of the first membrane and the second membrane comprises a semi-permeable portion configured to regulate diffusion characteristics of glucose through the membrane to realize a sensitivity for a sensor in the cavity.

In yet another aspect, a method is provided for encasing a biosensor. The method includes: providing a first membrane with an interlocking segment proximate a sensing element having an undercut width; depositing a second membrane into the interlocking segment and over the first membrane and the sensing element; and interlocking the second membrane with the first membrane along the interlocking segment to mitigate edge lifting of the first membrane relative to the second membrane.

These and other aspects are contemplated and described herein. It will be appreciated that the foregoing summary sets out representative aspects of a system and method for detecting releasable coupling of a cap with an insulin delivery device and assists skilled readers in understanding the following detailed description.

### BRIEF DESCRIPTION OF THE DRAWINGS

FIG. 1 is a perspective view from above of a glucose sensor encasement with a substrate membrane layer having an interlocking trench pattern in a base insulation layer for encasing an in-vivo sensor to mitigate membrane layer peeling proximate electrodes of a glucose limiting membrane interlock with the base insulation layer.

FIG. 2 is a plan view of the substrate membrane layer of FIG. 1 with the top layer and sensor removed.

FIG. 3 is a vertical sectional view taken along line 3-3 of FIG. 2.

FIG. 4 is a perspective view from above of another glucose sensor encasement having a slightly modified substrate membrane over that shown in FIGS. 1-3 with an interlocking trench pattern in a base insulation layer, a glucose limiting membrane interlocked with the base insulation layer, and a sensing electrode encased between the interlocked membrane layers.

FIG. 5 is a plan view of the substrate membrane layer of FIG. 4.

FIG. 6 is a vertical sectional view taken along line 6-6 of FIG. 5.

FIG. 7 is a mask plan view of one GOx sensor having a glucose sensing encasement of FIGS. 1-6.

FIG. 8 is an optional glucose sensing encasement over that shown in FIG. 7.

FIG. 9 is yet another optional glucose sensing encasement over that shown in FIGS. 7 and 8.

FIG. 10 is even another optional glucose sensing encasement over that shown in FIGS. 7-9 showing a discrete spaced-apart array of interlocking segments forming a zipper proximate each sensing electrode.

FIG. 11 is yet even another optional glucose sensing encasement over that shown in FIGS. 7-10 showing a discrete spaced-apart array of interlocking segments forming a zipper proximate each sensing electrode.

### DETAILED DESCRIPTION OF THE EMBODIMENTS

This disclosure is submitted in furtherance of the constitutional purposes of the U.S. Patent Laws “to promote the progress of science and useful arts” (Article 1, Section 8).

As used herein, the terms “portion” and “segment” refer to any structure capable of providing an interlocking interface along a discrete span or an encircling span that joins unto itself, such as a circle, oval, square, rectangle or other suitable geometric shape capable of substantially adjoining or encompassing a sensor region, and can also include discrete enlarged head posts and complementary entrapping ports.

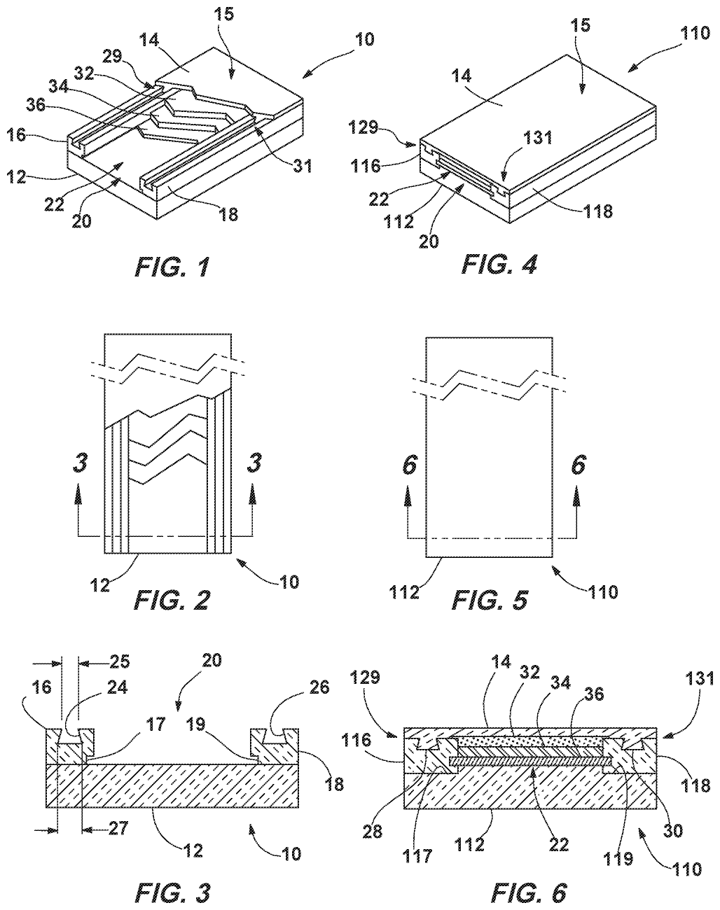

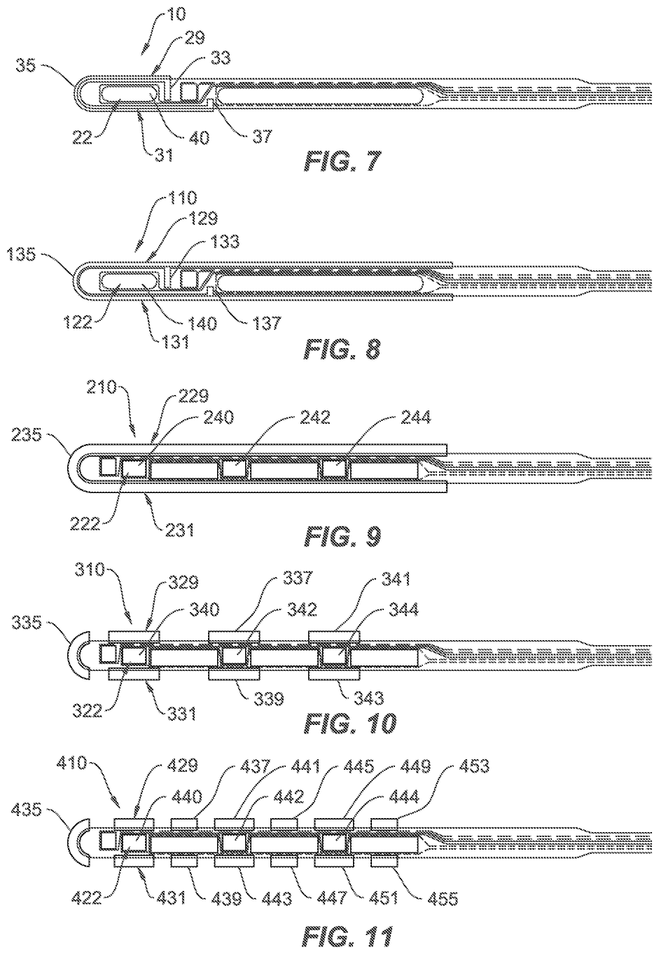

FIG. 1 illustrates a glucose sensor encasement having a substrate layer, or membrane 12 interlocked with a glucose limiting membrane (GLM) 14 according to one aspect. Membrane 14 comprises a semi-permeable portion 15 configured to regulate diffusion characteristics of glucose through membrane portion 15 to realize a sensitivity for a sensor in a cavity, or trench 20. Layer 12 supports a pair of spaced-apart and interlocking segments, or elongate dovetail rails 16 and 18. Elongate cavity, or trench 20 is provided between rails 16 and 18 configured to receive a sensor 22 comprising one or more sensing elements, or electrodes as depicted in FIGS. 7-11, such as GOx sensor 22 of FIG. 7 and is formed from deposited mask layers comprising a glucose oxidase enzyme layer 32, a platinum layer 34, and a gold layer 36. Accordingly, a mechanical locking feature is provided between layers 12 and 14 that prevents peeling apart of such layers proximate a sensing electrode of sensor 22.

As used herein, any form of membrane can be dovetail joined to another surface or membrane in order to substantially encase or secure or adhere edges or local portions of such membranes from pulling apart during use, and does not necessarily need to be limited to use on sensor limiting membranes. It is also understood that raised portions can be substituted for depressions, or grooves (and vice versa) when implementing such dovetail mechanical locking feature between two or more membranes.

FIG. 3 illustrates a vertical sectional view taken along line 3-3 of FIG. 2 one suitable geometry of substrate layer 12 of encasement 10 with layer 14 and sensor 22 removed. As shown in FIG. 3, dovetail interlocking segments, or rails 16 and 18 are affixed to an insulation polymer layer, or substrate layer 12. Optionally, such segments 16 and 18 can be integrally formed on top of layer 12. Either way, an elongate undercut structural feature is formed in segments 16 and 18 represented as a dovetail having an ensmalled width 25 distal of layer 12 and an enlarged width 27 proximal layer 12. A sensor 22 (see FIG. 4) is then deposited in layers atop layer 12 and between segments 16 and 18 (as shown in FIGS. 4-6) after which a top layer, or membrane 14 is deposited atop and within rails 16 and 18 to interlock and encase sensor 22 between layers 12 and 14. According to one construction layer 12 and segments 16 and 18 are constructed from polyimide, an insulation layer patterned to receive one of the mask layout sensor designs depicted in FIGS. 7-11, such as sensor 22 in FIG. 7.

As shown in FIG. 3, layer 12 can be deposited in a first mask layer after which segments 16 and 18 are deposited on top of layer 12 in a second mask layer that also includes an undercut mechanical interlock 17 and 19 configured to entrap layer 36 onto layer 12. An epoxy or acrylic flexible adhesive can be added to layer, for example, layer 12 can be a polyimide layer configured as a coverlay having an adhesive layer. Segments 16 and 18 can also be made of polyimide. Optionally, to reduce alignment variation and simplify the process, segments 16 and 18 can be added to the insulation pattern mask layer for membrane 12 so that segments 16 and 18 are aligned in recessed channels 17 and 19 and layer 12 and rails 16 and 18 form a monolithic layer.

As shown in FIG. 4, glucose biosensor encasement 10 is shown assembled together with a glucose limiting membrane (GLM) 14 interlocked with insulation layer 12 on opposed edges along sensor 22 using opposed interlocking elongate segment assemblies 29 and 31. Membrane 14 comprises a semi-permeable membrane layer that forms a chamber for at least one sensing element to hold a reactant that reacts with an analyte to measure concentration of a reaction product with the sensing element, or sensor 22. The semi-permeable membrane allows passage of the analyte into the chamber where byproducts of a reaction can be detected and quantified. Each assembly 29 and 31 includes an interlocking dovetail 16 and 18 and an entrapped elongate tenon, or ridge 28 and 30 (see FIG. 3) that provides a physical, or mechanical interlock between layers in order to prevent any leakage along the edges and adjacent sensing elements that would otherwise make such byproduct detection inaccurate, such as when detecting glucose levels.

As detailed in FIG. 5, FIG. 6 shows the interlocked assembly in vertical sectional view through encasement 10 and internal sensor 22. Sensor 22 is provided in an assembled mask deposition layer configuration comprising a glucose oxidase enzyme layer 32, a platinum layer 34, and a gold layer 36. Deposition of layer 14 into dovetail segments 16 and 18 locks edges of layers 12 and 14 together adjacent to sensor 22 which prevents peeling of layers 12 and 14 in use in vivo within a patient. Such peeling otherwise reduces accuracy of sensor 22.

Although a classic dovetail geometry is provided within interlocking segments 16 and 18, it is understood that any cross-sectional geometry that has an ensmalled distal portion and an enlarged proximal portion (relative to layer 12) will provide such an interlocking physical geometry suitable to prevent peeling of layers 12 and 14, such as a cylindrical or elliptical elongate groove shape sized to entrap a complementary male elongate rope, or tenon portion, and can include octagonal, hexagonal, triangular, or any other interlocking geometry grooves and complementary tenons.

As shown in FIG. 6, glucose biosensor encasement 10 includes a first membrane 112 and a second membrane 14. First membrane 112 has a first interlocking segment 116 and 118. Second membrane 14 has a second interlocking segment 28 and 30 cooperating with the first interlocking segment 116 and 118 of the first membrane 112 to provide a cavity 20 (see FIGS. 1 and 4) between first membrane 112 and second membrane 14 for receiving a glucose sensor 22. Interlocking segment pairs 129 and 131 are provided when segments 28 and 30 are deposited within segments 116 and 118 respectively during a semiconductor/mask deposition operation. At least one of first membrane 112 and second membrane 14 comprises a semi-permeable portion 15 (see FIG. 4) configured to regulate diffusion characteristics of glucose through membrane 14 to realize a sensitivity for sensor 22 in cavity 20. A pair of elongate recess slots 117 and 119 are formed in membrane, or layer 112 to receive each interlocking segment 116 and 118 in alignment when deposited atop layer 112 during a deposition step from a mask operation.

FIG. 7 is a mask plan view of one glucose oxidase enzyme layer GOx sensor having a glucose sensing encasement 10 shown in simplified form in FIGS. 1-6. More particularly, glucose sensing encasement 10 is illustrated as a mask layup having a sensor 22 provided on an electrically conductive trace including a glucose oxidase enzyme sensing element, or electrode 40. Sensing element 40 is substantially encompassed by a circuitous and contiguous array of interlocking segment pairs including linear elongate interlocking segment pairs 29 and 31, semi-circular interlocking segment pair 35 extending between pairs 29 and 31, and a pair of transverse partial bulkhead interlocking segment pairs 33 and 37. Bulkhead segment pairs 33 and 37 provide just enough clearance for conductive traces on sensor 22 to communicate from sensor 40 with a power supply and data gathering computer having processing circuitry and memory (not shown).

FIG. 8 is an optional glucose sensing encasement 110 over that shown in FIG. 7. More particularly, encasement 110 includes linear elongate interlocking segment pairs 129 and 131 each contiguous at one end with a u-shaped interlocking segment pair 135 and a pair of partial bulkhead interlocking segment pairs 133 and 137 substantially encircling sensing element 140 of sensor 122 while providing a path for conductive traces to exit the substantially encircled region of sensing element 140.

FIG. 9 is yet another optional glucose sensing encasement 210 over that shown in FIGS. 7 and 8. More particularly, encasement 210 includes linear elongate interlocking segment pairs 229 and 231 each contiguous at one end with a u-shaped interlocking segment pair 235 substantially encircling, or proximately interlocking the top and bottom membranes adjacent the individual sensing elements 240, 242, and 244 of sensor 222 while providing a path for conductive traces to exit the substantially encircled region of sensing element 240 by exiting the open end of a long u-shaped interlocking pair of elongate u-shaped elements.

FIG. 10 is even another optional glucose sensing encasement 310 over that shown in FIGS. 7-9 and including interlocking segment pairs 329, 331, 335, 337, 339, 341, and 343 forming a staggered pair of adjacent interlocking segment pairs of lateral sides of each sensing element 340, 342, and 344 of sensor 322 to provide a zipper-like interlocking array of segment pairs proximate each sensing electrode. More particularly, interlocking segment pairs are provided along each sensor electrode.

FIG. 11 is yet even another optional glucose sensing encasement 410 over that shown in FIGS. 7-10 and including interlocking segment pairs 429, 431, 435, 437, 439, 441, 443, 445, 447, 449, 451, and 453, 455 forming a staggered pair of adjacent interlocking segment pairs of lateral sides of each sensing element 440, 442, and 444 of sensor 422 to provide a zipper-like interlocking array of segment pairs proximate each sensing electrode.

Segment pairs 437, 439, 445, 447, and 453, 455 of FIG. 11 are spaced adjacent, but offset from sensor elements 440, 442 and 442, 444 showing a discrete spaced-apart array of interlocking segments forming a zipper that further edge supports the top and bottom membranes covering the sensing electrode 440, 442, and 444. More particularly, interlocking segment pairs are provided along each sensor electrode, and further interlocking segment pairs are provided between adjacent electrodes in order to lock the top and bottom membranes, or layers together and prevent peeling of top layer 14 from bottom layer 12 (see FIG. 1) that would otherwise negatively influence side diffusion of glucose as used in-vivo.

The terms “a”, “an”, and “the” as used in the claims herein are used in conformance with long-standing claim drafting practice and not in a limiting way. Unless specifically set forth herein, the terms “a”, “an”, and “the” are not limited to one of such elements, but instead mean “at least one”.

While the subject matter of this application was motivated in addressing a glucose biosensor encasement, it is in no way so limited. The disclosure is only limited by the accompanying claims as literally worded, without interpretative or other limiting reference to the specification, and in accordance with the doctrine of equivalents. Other aspects and implementations of other biosensor encasements are contemplated.

In compliance with the statute, the various embodiments have been described in language more or less specific as to structural and methodical features. It is to be understood, however, that the various embodiments are not limited to the specific features shown and described, since the means herein disclosed comprise disclosures of putting the various embodiments into effect. The various embodiments are, therefore, claimed in any of its forms or modifications within the proper scope of the appended claims appropriately interpreted in accordance with the doctrine of equivalents.

1. A method for encasing a biosensor, comprising:
providing a first membrane with a first interlocking segment proximate a sensing element having an undercut width;
depositing a second membrane into the first interlocking segment and over the first membrane and the sensing element; and
interlocking the second membrane with the first membrane along the first interlocking segment to mitigate edge lifting of the first membrane relative to the second membrane.

2. The method of claim 1, wherein the first interlocking segment of the first membrane comprises a dovetail segment.

3. The method of claim 1, further comprising substantially encompassing the sensing element with the interlocking segment.

4. The method of claim 1, wherein the second membrane comprises a semi-permeable portion, and further comprising regulating diffusion characteristics of glucose through the membrane to realize a sensitivity for the sensing element beneath the second membrane.

5. The method of claim 1, wherein the first interlocking segment comprises an elongate ridge.

6. The method of claim 5, further comprising forming a second interlocking segment.

7. The method of claim 6, wherein the second interlocking segment is formed to comprise a channel having an ensmalled distal width and an enlarged proximal width complementary to the elongate ridge and interlocking in assembly.

8. The method of claim 6, wherein the first interlocking segment and the second interlocking segment are each provided respectively proximate an outer periphery of the first membrane and the second membrane.

9. The method of claim 1, wherein the first membrane and the second membrane cooperate to provide a plurality of spaced-apart sensor cavities, at least one pair of first interlocking segment and second interlocking segment provided proximate each sensor cavity

10. The method of claim 6, wherein a plurality of discrete first interlocking segments and a complementary plurality of discrete second interlocking segments are provided spaced apart about an outer periphery of the cavity between the first membrane and the second membrane.
